(19) United States
(12) Patent Application Publication
(10) Pub. No.: US 2024/0023864 A1
(43) Pub. Date: Jan. 25, 2024

(54) ELECTRODE DEVICE AND RELATED METHODS

(71) Applicant: UNIVERSITY OF SOUTH FLORIDA, Tampa, FL (US)

(72) Inventors: Stephen Edward SADDOW, Tampa, FL (US); Chenyin FENG, Hubei (CN); Christopher Leroy FREWIN, Monroe, MI (US)

(21) Appl. No.: 18/263,466

(22) PCT Filed: Jan. 31, 2022
(86) PCT No.: PCT/US2022/014632

§ 371 Date: Jul. 28, 2023

Related U.S. Application Data
(60) Provisional application No. 63/199,874, filed on Jan. 29, 2021.

(51) Int. Cl. A61B 5/263; A61N 1/05

(52) U.S. Cl. CPC A61B 5/263; A61N 1/0529

(57) ABSTRACT

The present disclosure provides an electrode device and methods of making and using the same. The electrode device includes a base layer, an intermediate layer, and a capping layer. Both the intermediate layer and the capping layer are located over the base layer. The intermediate layer includes a carbon-based electrode. The base layer and the capping layer each include silicon carbide. The capping layer partially surrounds the carbon-based electrode. The method of using the electrode device as an implantable neural interface involves providing the electrode device, electrically coupling the carbon-based electrode to neural tissue of a patient, and electrically coupling the carbon-based electrode to at least one of recording electronics and stimulating electronics. The recording electronics are configured to electrically record neural signals from the neural tissue, whereas the stimulating electronics are configured to electrically stimulate the neural tissue.

### CROSS-REFERENCE TO RELATED APPLICATIONS

The present application claims priority to U.S. Provisional Patent Application No. 63/199,874, filed Jan. 29, 2021, the entire disclosure of which is incorporated by reference herein.

### BACKGROUND

Implantable neural interfaces (INIs) are devices configured to connect with the human nervous system through electrical means. Advantageously, such devices allow electrical recording and stimulation of the nervous system and may contribute to developing effective treatments for various nervous system diseases (e.g., depression, addiction, Parkinson's, Alzheimer's, and poliomyelitis).

However, the materials used for conventional INIs (e.g., silicon and noble metals) suffer from many limitations. For example, due to a relatively low impedance and small detection window, noble metals and silicon-based INIs have limited application during neural stimulation. Additionally, long-term implantable performance with such devices is poor due to the foreign body response, loss of target neurons, and scar (i.e., gliosis) formation. Furthermore, noble metals introduce irreversible dissolution during neurostimulation, which can cause undesirable damage to the human body.

Accordingly, new implantable device materials for biomedical applications are desirable.

### SUMMARY

In accordance with some embodiments of the disclosed subject matter, an electrode device, method of making the same, and method of using the electrode device as an implantable neural interface are provided.

In accordance with some embodiments of the disclosed subject matter, an electrode device comprises a base layer, an intermediate layer, and a capping layer. Both the intermediate layer and the capping layer are located over the base layer. The base layer and the capping layer each comprise silicon carbide. The intermediate layer comprises a carbon-based electrode, and the capping layer partially surrounds the carbon-based electrode.

In some embodiments, the carbon-based electrode comprises graphene, graphene oxide, reduced graphene oxide, carbon nanotubes, and/or a pyrolyzed-photoresist-film.

In certain preferred embodiments, the carbon-based electrode comprises (e.g., is) a pyrolyzed-photoresist-film (PPF). Such embodiments represent an improvement over conventional electrodes since they replace the metal traces of the electrode with pyrolyzed-photoresist-film. Advantageously, such embodiments reduce manufacturing costs and simplify the manufacturing process as compared to conventional electrodes.

In preferred embodiments, the silicon carbide of both the base layer and the capping layer comprises amorphous silicon carbide. In other cases, the silicon carbide of the capping layer is amorphous, but the silicon carbide of the base layer is not amorphous.

In some embodiments, the capping layer of the fabricated electrode device has a thickness in a range of from 0.5 μm to 5 μm.

In some embodiments, the base layer of the fabricated electrode device has a thickness in a range of from 0.5 μm to 5 μm.

In some embodiments, the intermediate layer of the fabricated electrode device has a thickness in a range of from 0.1 μm to 1 μm.

In some embodiments, a metal contact pad is electrically coupled to the carbon-based electrode through carbon traces (e.g., PPF traces). When the electrode device is in its fully-fabricated form, the metal contact pad is exposed through an opening in the capping layer.

Some embodiments provide a method of making the electrode device. The method comprises depositing a base layer onto an electronic wafer; forming an intermediate layer on the base layer; depositing a metal layer onto carbon (e.g., PPF) traces; and depositing a capping layer onto the intermediate layer. The base layer and the capping layer each comprise silicon carbide, the intermediate layer comprises a carbon-based electrode (e.g., PPF), and the metal layer comprises a metal contact pad. The carbon traces are configured to electrically couple the electrode to the metal contact pad. The method further includes forming one or more openings in the capping layer to expose at least a portion of the carbon-based electrode (e.g., to expose the recording and wire-bonding sites). In some embodiments, the method also includes releasing the electronic wafer from the base layer to form a free-standing probe/electrode device.

In some embodiments, the step of forming an intermediate layer on the base layer comprises depositing a photoresist onto the base layer, patterning the photoresist to a desired shape, and pyrolyzing the photoresist so as to convert the photoresist to a pyrolyzed photoresist film.

In some embodiments, the step of releasing the electronic wafer involves subjecting the electronic wafer to an acid bath or a solvent bath.

Some embodiments provide a method of using the electrode device as an implantable neural interface. The method involves providing the electrode device. The electrode device comprises a base layer, an intermediate layer, and a capping layer. Both the intermediate layer and the capping layer are located over the base layer. The base layer and the capping layer each comprise silicon carbide. The intermediate layer comprises a carbon-based electrode, and the capping layer partially surrounds the carbon-based electrode. The method also includes electrically coupling the carbon-based electrode to neural tissue of a patient. Still further, the method includes electrically coupling the carbon-based electrode to at least one of recording electronics and stimulating electronics. The recording electronics are configured to electrically record neural signals from the neural tissue, whereas the stimulating electronics are configured to electrically stimulate the neural tissue.

Some embodiments further comprise electrically recording neural signals from the neural tissue using the recording electronics.

Some embodiments further comprise electrically stimulating the neural tissue using the stimulating electronics.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 is a cross-sectional view of an electrode device in accordance with certain embodiments of the present disclosure.

FIG. 2A shows biocompatibility of α-SiC fluorescent images from deep cortical tissue with α-SiC-coated probe (left image) and Si probe (right image) inserted.

FIG. 2B shows a plot of glial fibrillary acidic protein (GFAP) intensity with distance from the insertion site for both the α-SiC and Si probes illustrated in FIG. 2A.

FIG. 2C shows comparison data of platelet attachment to Si, 3C—SiC, SiO₂, and α-SiC. The results show that 3C- and α-SiC displayed a lower concentration of platelets bound to the surface than Si and SiO₂, indicating that 3C- and α-SiC are more hemocompatible.

FIG. 3A illustrates an example graphene electrode for neural imaging and optogenetic device.

FIG. 3B shows cell morphology from a confocal laser scanning microscope (CLSM) after cell growth on GO/PEDOT electrodes.

FIG. 3C shows spinal cord cell growth and reconnection on 3D CNT foam.

FIG. 3D is a graph showing results from in vitro cell viability assays comparing the viability of human keratinocyte (HaCaT) cells on 6H—SiC, graphene, and PPF.

FIG. 4 shows top views of two example electrode devices in accordance with certain embodiments of the present disclosure.

FIG. 5 illustrates an example of a process for making an electrode device in accordance with certain embodiments of the present disclosure.

FIG. 6A illustrates an example process of using mechanical exfoliation to obtain graphene.

FIG. 6B illustrates an example process of using chemical vapor deposition (CVD) to obtain graphene.

FIG. 6C illustrates epitaxial growth graphene on SiC.

FIG. 6D illustrates an example process to obtain laser-induced graphene.

FIG. 7 shows an example process for synthesizing graphene oxide using Modified Hummer's method. Bulk graphite powder transforms into few-layer graphene flakes and is oxidized into graphene oxide (GO), as shown via wet chemical processing.

FIG. 8 shows an example technique for fabricating a pyrolyzed-photoresist-film (PPF) electrode on amorphous SiC according to some embodiments of the present disclosure. Steps 1-9 are cross-sectional views of an exemplary fabrication process of the α-SiC/PPF device, with step 0 illustrating a top view of an example PPF mINP shank according to certain exemplary embodiments of the present disclosure.

FIG. 9A is a plan-view SEM image of an α-SiC/PPF free-standing probe, with a scale bar of 1 mm (50 μm for inset).

FIG. 9B is a cross-sectional SEM image of the PPF sample show in FIG. 9A before annealing, with a scale bar of 500 nm. The initial photoresist thickness was 7.4 μm prior to annealing at 700 degrees Celsius.

FIG. 9C shows Raman spectra of PPF annealed versus temperature.

FIG. 9D shows electrochemical data versus recording area, with impedance of about 10 kΩ at 1 kHz for all electrodes tested.

FIG. 10 illustrates an example of a process of using an implantable neural interface/neural probe in a patient in accordance with the subject matter disclosed herein.

FIG. 11 shows an example of hardware that can be used to implement a computing device according to various embodiments described herein.

The drawings illustrate only example embodiments and are therefore not to be considered limiting of the scope of the embodiments described herein, as other embodiments are within the scope of the disclosure.

### DETAILED DESCRIPTION

Carbon is one of the most versatile elements in the periodic table. It can covalently bond with itself or with other elements because it has four bonds that can form into different compounds and forms. Furthermore, there are many allotropes of carbon, attributed to the different hybridized carbon electron orbitals (e.g., sp, sp², and sp³).

Nanomaterials and nanotechnology can be useful in certain biomedical applications due to their ability to control and modulate the electrical, mechanical, and chemical properties of devices fabricated at the micro- and nanoscale levels. Many carbon-based nanomaterials exhibit excellent electrical and biocompatible properties when they interact with the bio-environment.

In accordance with various embodiments, this disclosure provides an electrode device that can be used as a chronic (i.e., long-term) implantable neural interface. More particularly, and as described in more detail below, the present disclosure uses carbon-based nanomaterials (CBNs) (e.g., graphene, graphene oxide, reduced graphene oxide, carbon nanotubes, and/or pyrolyzed-photoresist-film) as the conductive layer for implantable neural interfaces.

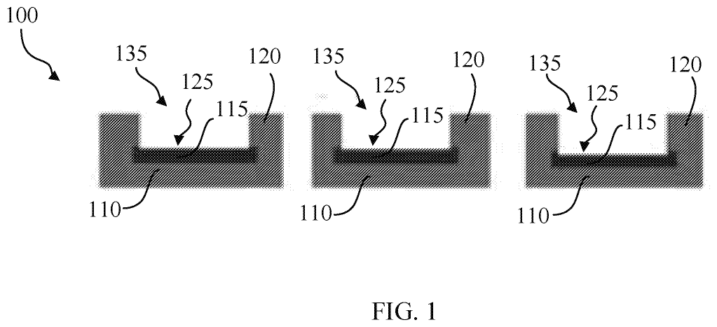

FIG. 1 shows the electrode device 100 of the present disclosure. The electrode device 100 comprises a base layer 110, an intermediate layer 115, and a capping layer 120. The base layer 110 serves as the supporting layer of the electrode device 100. The intermediate layer 115 and the capping layer 120 are each located over the base layer 110. As described in more detail below, the capping layer 120 either partially or entirely surrounds the intermediate layer 115, depending on the particular stage of the process for fabricating the electrode device 100.

The base layer 110 and the capping layer 120 of the electrode device 100 each comprise silicon carbide. Silicon carbide is a semiconductor material which has more than 250 polymorphs, exhibits high tolerance to harsh environments, including long-term stability in acid/base solutions, at high temperatures, and in high radiation environments. These excellent properties give SiC the ability to be utilized as an INI material.

In certain preferred embodiments, the silicon carbide of both the base layer 110 (i.e., the substrate) and the capping layer 120 comprises amorphous silicon carbide. In such cases, the amorphous form, α-SiC, was chosen as the base layer 110 and capping layer 120 for the electrode because of its robust chemical inertness, excellent insulating properties, and observed biocompatibility. In other cases, the silicon carbide of the capping layer is amorphous, but the silicon carbide of the base layer is not amorphous.

In some embodiments, the capping layer of the fabricated electrode

Significantly, the biocompatibility and hemocompatibility of α-SiC has been demonstrated in prior studies and is described in the following literature:
1—Saddow S E, et al (2014), 3C—SiC on Si: a bio- and hemo-compatible material for advanced nano-bio devices. In: 2014 IEEE 9th Nanotechnol. Mater. Devices Conf. NMDC, pp. 49-53, https://doi.org/10.1109/NMDC.2014.6997419.
2—Knaack G L, Charkhkar H, Cogan S F, Pancrazio J J (2016), Chapter 8: amorphous silicon carbide for neural interface applications, Second edn. Elsevier Inc., Amsterdam, p. 2016.
3—Beygi M et al (2019), Fabrication of a monolithic implantable neural interface from cubic silicon carbide. Micromachines 10(7):430. https://doi.org/10.3390/mi10070430.

Results from at least some of these references are briefly summarized in the following three paragraphs.

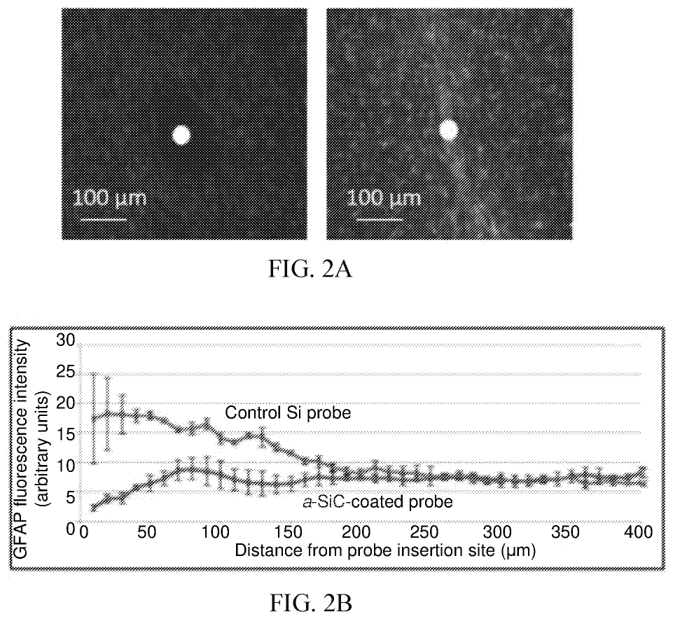

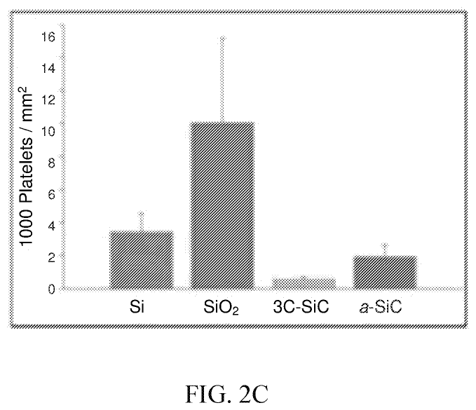

To investigate the chronic implantation of α-SiC, a control Si probe along with the α-SiC probe was implanted in the same rat brain for four weeks. FIG. 2A shows immunohistochemically stained horizontal slices from an α-SiC and Si implant. The white circle indicates the probe locations, glial fibrillary acidic protein (GFAP) labeling in green, and the deep cortical tissue is labeled in red. By comparing the α-SiC-coated probe with the control Si probe, the α-SiC-coated probe showed a significant decrease in GFAP intensity at the insertion site, which means a decreased level of activated astrocytes.

FIG. 2B shows the summary data for this prior work of GFAP intensity with distance from the insertion site for both the α-SiC and Si probes. The hemocompatibility was examined by the number of attached platelets to the material. A lower concentration of attached platelets indicates that the material is more hemocompatible since platelets will trap blood cells and form a thrombus. In a separate work, all samples were exposed to platelet-rich plasma for 15 min afterward the presence of attached platelets was studied by fluorescent microscopy.

Comparison data of platelet attachment to Si, 3C—SiC, SiO₂, and α-SiC is shown in FIG. 2C. The results indicate that 3C—SiC has the lowest platelet binding followed by α-SiC which displayed slightly higher platelet binding than 3C—SiC, whereas SiO₂ and Si had a much higher number of bound platelets. This result further indicates that Si and SiO₂ are not suitable for biomedical applications, especially when the devices are in contact with the vascular system.

The intermediate layer 115 of the electrode device 100 comprises a carbon-based electrode 125. As used herein, a “carbon-based electrode” refers to an electrode that includes carbon-based materials. In some cases, the carbon-based electrode 125 comprises graphene, graphene oxide, reduced graphene oxide, carbon nanotubes, and/or a pyrolyzed-photoresist-film. In some embodiments, the carbon-based electrode 125 consists of, or consists essentially of, the materials noted in the preceding sentence. Skilled artisans will appreciate that the list of materials identified in this paragraph is by no means limiting, and other carbon-based materials can also be used for the carbon-based electrode 125.

Graphene has many excellent intrinsic properties, including atomic-scale thickness, structurally lightweight at 0.77 mg/m² high tensile stiffness (˜10³ GPa), high heat conductivity (5000 W/mK) at room temperature, and very high carrier mobility of 15,000 cm²v⁻¹s⁻¹. Additionally, graphene has shown biocompatibility results in vitro.

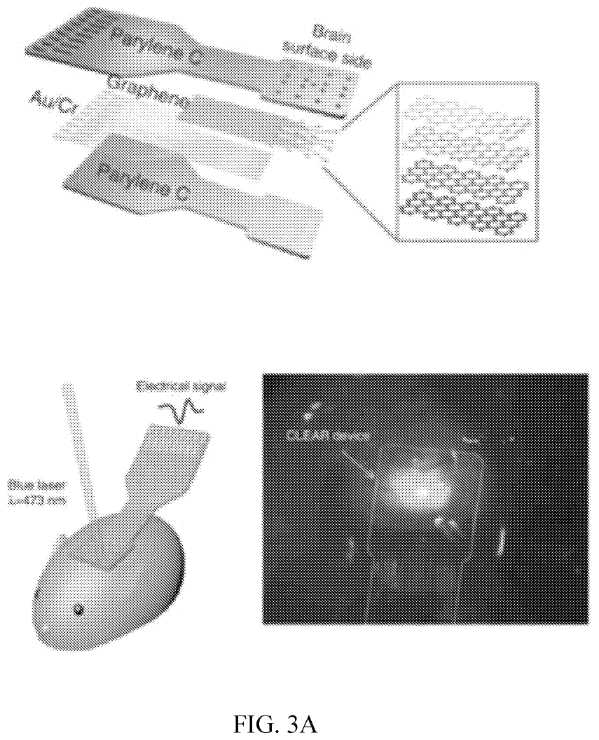

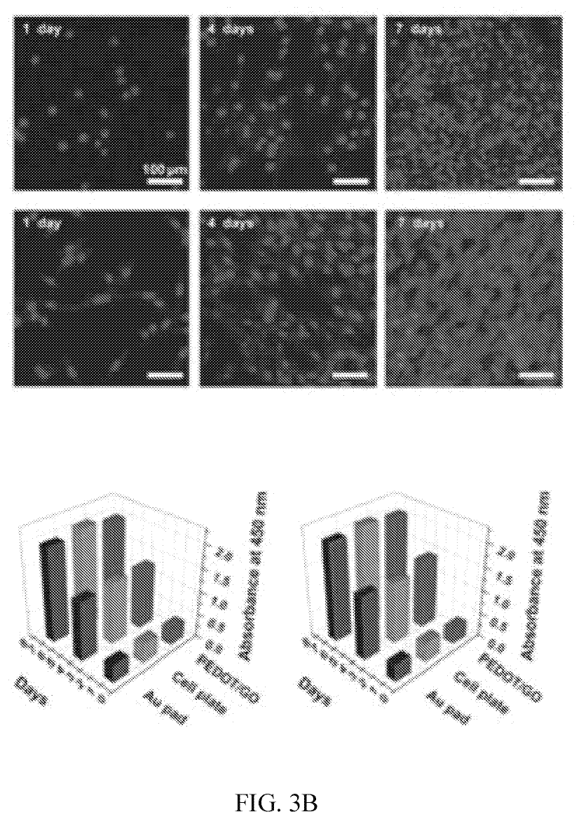

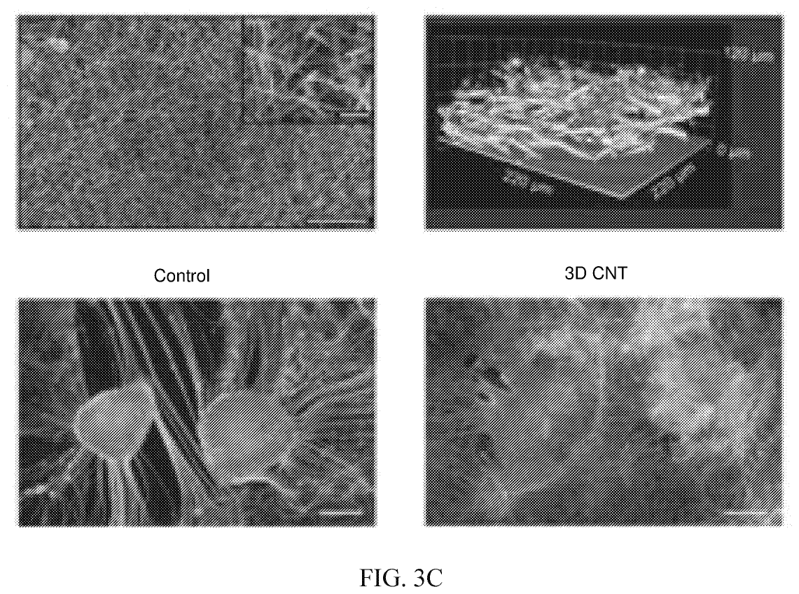

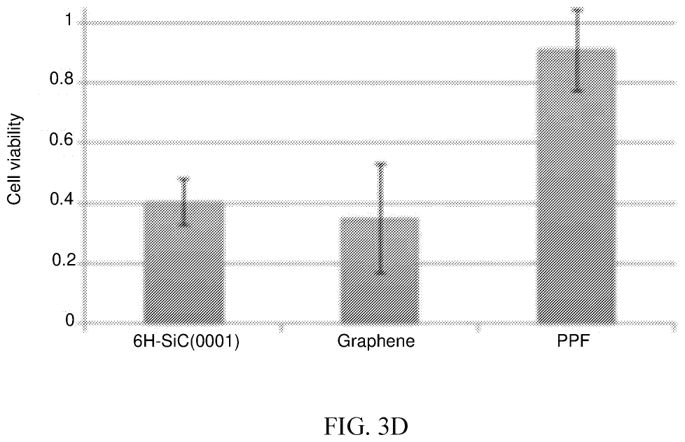

Graphene electrodes have also shown long-term biocompatibility. For example, Park and collaborators fabricated a carbon-layered electrode array (CLEAR) based on graphene and Parylene C, as shown in FIG. 3A. The optical transparency of this device was characterized at >90% transmission from the ultraviolet to the infrared part of the optical spectrum. Advantageously, this device was used for neurophysiological recording and to achieve optogenetic activation of cortical areas under the electrodes and imaging of blood vasculature by fluorescence microscopy at the same time. In addition, this research demonstrated that graphene substrates can promote neurite sprouting and outgrowth, which indicates that graphene can reduce the tissue inflammatory response.

Furthermore, graphene-derived materials such as graphene oxide (GO) and reduced graphene oxide (rGO) have also proven to be suitable for biomedical and bioelectrical applications. Tian et al. reported a GO doped poly(3,4-ethylenedioxythiophene) (PEDOT) hybrid film on a gold wire electrode, which showed enhanced mechanical and electrochemical impedance performance. The impedance at 1 kHz of GO/PEDOT gold electrode demonstrated a two order of magnitude decrease compared with bare gold electrodes. In addition, they also illustrated the biocompatibility of GO/PEDOT coated gold electrode using PC-12, and NIH/3′T3 cells which were cultured on GO/PEDOT electrodes for several days to investigate the cell proliferation.

FIG. 3B shows cell morphology from a confocal laser scanning microscope (CLSM) after cell growth versus time. These results indicate that both cell lines thrive on GO/PEDOT electrodes, demonstrating that the system neither facilitates nor restrains cell proliferation compared with gold pads and cell plates and indicates that GO/PEDOT has excellent biocompatibility such as gold and cell culture plates.

Carbon nanotubes (CNTs) exhibit excellent electrical, mechanical, and thermal transfer properties. Carbon nanotubes can be made of single or multilayer graphene rolled into a cylindrical shape. The diameter and wall layer of the CNT can be tuned by controlling various parameters during synthesis. Because of the high surface area, great mechanical properties, and excellent chemical and thermal stability, CNTs are a promising material for bio-electronic devices. 3D CNT foam (CNF) can help spinal cord cell growth and reconnection, as shown in FIG. 3C. The top left image shows an SEM image of the CNF, and the top right is the confocal 3D reconstruction of this CNF. The bottom two images show two weeks ex vivo cultured spinal organotypic slices in the control group and CNF group. It shows a significant outgrowth of nerves in both groups.

In certain exemplary embodiments, the carbon-based electrode 125 comprises a pyrolyzed-photoresist-film. Advantageously, such electrodes replace the metal traces of a conventional electrode with a pyrolyzed-photoresist-film. This in turn reduces manufacturing costs and simplifies the manufacturing process as compared to conventional electrodes. A pyrolyzed-photoresist-film can optionally be provided in cases where the capping layer 120 and the base layer 110 both comprise amorphous silicon carbide. Such an embodiment provides a highly robust, thin, and flexible neural interface using neuro-compatible materials. A flexible material is advantageous as it enables the electrode device 100 to move smoothly with the neural tissue of a patient when the electrode device 100 is implanted into neural tissue of the patient. Pyrolyzed-photoresist-film also possesses excellent preliminary electrochemical properties and does not require assistive devices to implant into the tissue. In such embodiments, the carbon-based electrode 125 can optionally consist of pyrolyzed-photoresist-film, while the capping layer and the base layer can optionally consist of amorphous silicon carbide.

Photoresists are a type of polymer resin that contains photoactive compounds and is an essential material for integrated circuit (IC) fabrication. One advantage of using PPF for biomedical applications is that it can enable facile patterning by standard photolithography processes and then be converted to a conductive carbon film via thermal annealing. In addition, PPF is electrically conductive, chemically inert, and biocompatible. FIG. 3D shows results of a study demonstrating that PPF can promote cell viability. Human keratinocyte (HaCaT) cells were used for cell viability testing, while graphene and 6H—SiC were used as a comparison group. The HaCaT cells were seeded with a density of 30,000 cells/cm². After 72 h incubation, the viability value of HaCaT cells was 0.35±0.18 (standard deviation of the mean), 0.41±0.08, and 0.91±0.13 for graphene, 6H—SiC, and PPF, respectively. This study illustrated that PPF is more biocompatible than graphene and 6H—SiC and thus a possible candidate for implantable devices.

Various properties of certain CBNs are been summarized in Table 1 below.

TABLE 1
Properties of Carbon-Based Nanomaterials

| | Electron | Young's | Electrical | |
|---|---|---|---|---|
| Carbon | mobility | modulus | conductivity | No. of |
| material | (cm²/V s) | (GPa) | (S/m) | Dimensions |
| Graphene | 15,000 | 856 | 10⁷-10⁸ | 2 |
| CNT | 15,000 | 1000 | 10⁶-10⁷ | 1 |
| PPF | — | — | — | 3 |
| 3C-SiC^{a} | 800 | 433 | ~10^{4b} | 3 |
| 4H-SiC^{a} | 1000 | 444 | ~10^{3b} | 3 |

^{a}n-doped material
^{b}Actual conductivity depends on doping density

In any embodiment of the present disclosure, the carbon-based electrode 125 can comprise a plurality of carbon-based electrodes (e.g., be provided as a micro-electrode array). As described in more detail below, this can be particularly useful since different electrodes can then be used for different purposes (e.g., stimulating neural tissue vs. recording neural signals).

In some embodiments, the base layer 110 and the capping layer 120 each have a thickness in a range of from 0.5 μm to 5 μm, such as from 0.5 μm to 4 μm, or from 0.5 μm to 2 μm, with each of these thicknesses referring to thickness values of the layers for the fabricated electrode device. In any embodiment of the present disclosure, the base layer 110 and the capping layer 120 can have the same thickness or can have different thicknesses from each other.

In certain embodiments, the intermediate layer 115 has a thickness in a range of from 0.1 μm to 1 μm (e.g., from 0.1 μm to 0.5 μm, or from 0.5 μm to 1 μm, or other subranges), with these thicknesses referring to thickness values of the layer for the fabricated electrode device. Any of these thickness ranges can be provided in combination with any of the thickness ranges for the base layer 110 and the capping layer 120 recited in the preceding paragraph. However, these thickness ranges are by no means limiting and alternative thicknesses can also be used. The thickness values selected will depend on the intended use of the device, desired electrical resistance, and other design considerations. As a non-limiting example, in some cases, the base layer 110 may have a thickness of 2 μm, the capping layer 120 may have a thickness of 0.5 μm, and the intermediate layer 115 may have a thickness of 0.5 μm (where each thickness is the thickness of the layer for the fabricated electrode device).

As used herein, the term “fabricated electrode device” refers to the electrode device in its fully-fabricated form (i.e., after annealing). Skilled artisans will appreciate that annealing during the fabrication process will cause many layers to shrink. Thus, the thickness of many of the layers will be much thicker as-deposited to account for shrinkage caused by the annealing process. In some cases, annealing may cause certain layers of the electrode device to shrink vertically by about 93% to about 95%. The present inventors found that lateral shrinking (due to annealing) was negligible, while the trace and electrode area of the device remained constant.

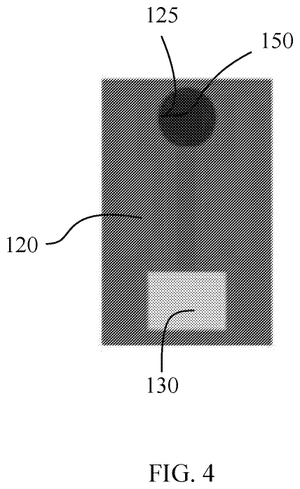

As shown in FIG. 4, the electrode device 100 further comprises a metal contact pad 130 electrically coupled to the carbon-based electrode 125 through the carbon (e.g., PPF) traces. During the fabrication process, the metal contact pad 130 becomes exposed through an opening in the capping layer 120. As explained in more detail below, this can be accomplished by etching the capping layer 120 to create one or more openings.

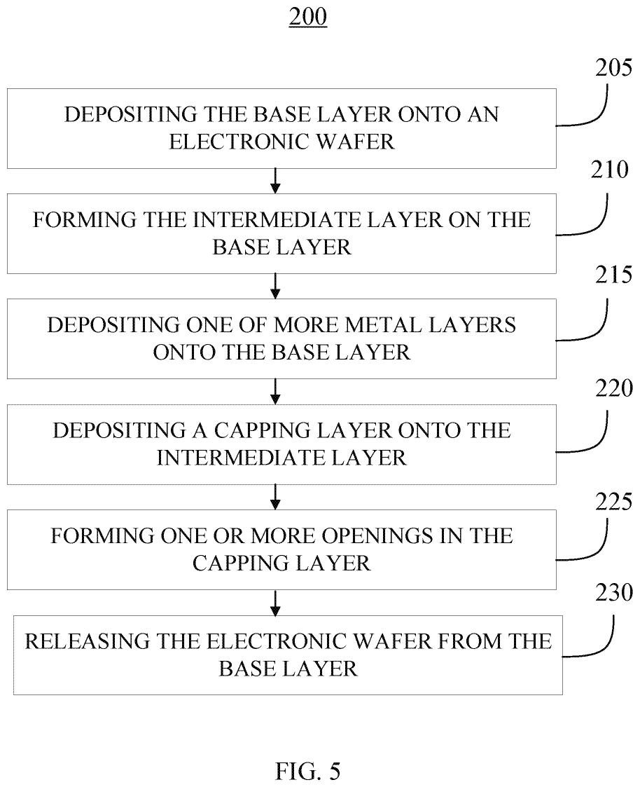

As shown in FIG. 5, the present disclosure also provides a method 200 of making an electrode device. The method 200 comprises the step 205 of depositing the base layer 110 onto an electronic wafer 140. As discussed above, the base layer 110 comprises silicon carbide. The electronic wafer 140 can comprise any known wafer material, such as silicon, germanium, or the like.

The method 200 also includes the step 210 of forming the intermediate layer 115 on the base layer 110. As discussed above, the intermediate layer 115 comprises a carbon-based electrode 125 (e.g., graphene, graphene oxide, reduced graphene oxide, carbon nanotubes, and/or a pyrolyzed photoresist film). In embodiments where the carbon-based electrode 125 comprises a pyrolyzed photoresist film, this step can involve depositing a positive photoresist 145 onto the base layer 110, patterning the photoresist 145 to a desired shape, and pyrolyzing the photoresist 145 so as to convert the photoresist 145 to a pyrolyzed photoresist film 150. In embodiments where the carbon-based electrode 125 comprises a different carbon-based material (i.e., other than pyrolyzed photoresist film), skilled artisans will appreciate that alternative deposition techniques would be performed instead.

Still further, the method 200 includes the step 215 of depositing one or more metal layers onto carbon (e.g., PPF) traces. The metal layer comprises (e.g., is) a metal contact pad (e.g., metal contact pad 130). The carbon traces are configured to electrically couple the electrode to the metal contact pad. In some cases, in a later step (i.e., after a step of annealing the metal contact pad), additional metal layers can be deposited over the metal contact pad to facilitate wire bonding.

The method 200 further includes depositing a capping layer 120 onto the intermediate layer 115 (step 220). As discussed above, the capping layer 120 comprises silicon carbide. The method further includes the step 225 of forming one or more openings in the capping layer 120 to expose least a portion of the carbon-based electrode 125 and at least a portion of the metal contact pad 130.

The method also includes the optional step 230 of releasing the electronic wafer 140 from the base layer 110. This step can involve any known silicon wet-etching techniques. For example, in some cases, the step 230 of releasing the electronic wafer 140 from the base layer 110 involves subjecting the electronic wafer 140 to an acid bath (e.g., a hydrofluoric acid bath) or a solvent bath. Skilled artisans will appreciate that alternative wet-etching techniques can be used instead.

Various deposition techniques for depositing layers of material are well-known in the art. The examples below describe certain deposition and fabrication techniques that can, but need not, be used to form the electrode device 100 of the present disclosure.

### Example 1—Methods for Graphene Growth

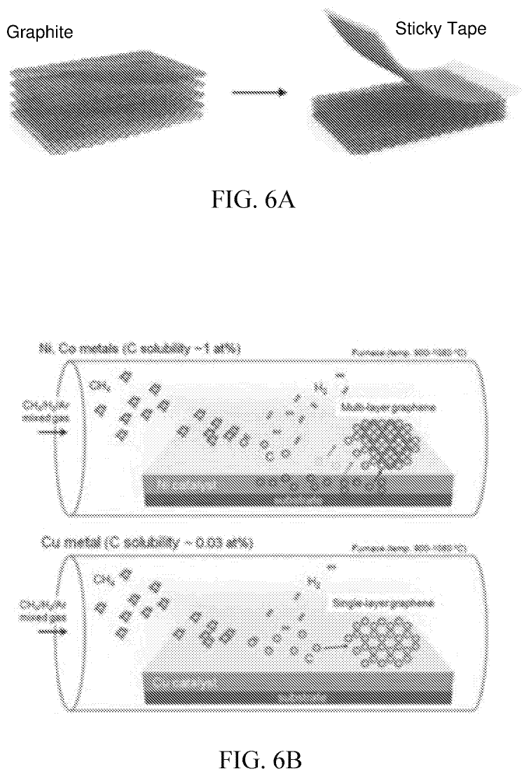

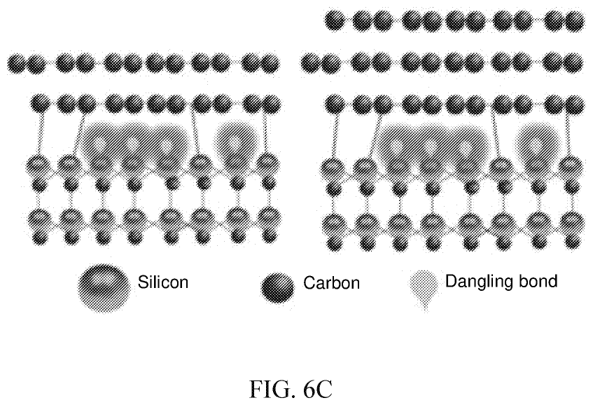

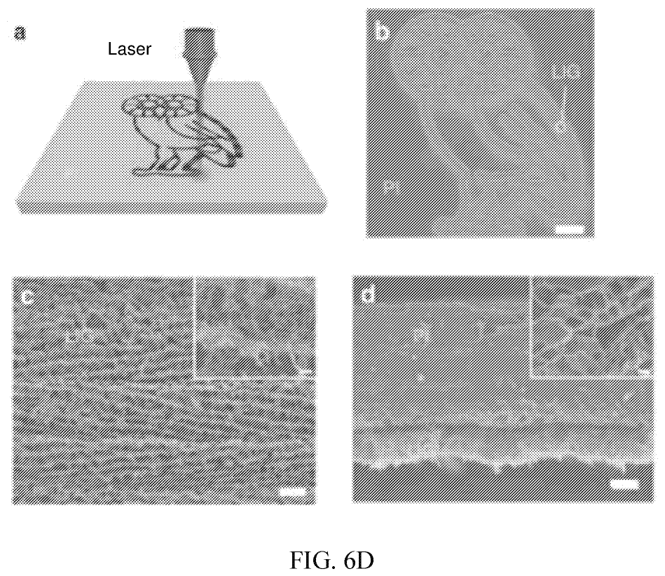

Various methods are contemplated for large scale fabrication of graphene such as mechanical, liquid, and gas exfoliation, chemical vapor deposition (CVD), epitaxial growth (EG) on silicon carbide, laser-induced graphene (LIG), etc. Mechanical exfoliation to obtain graphene is shown in FIG. 6A, CVD graphene, which is shown FIG. 613, is the most commonly used method because it can enable large-area growth, crystallinity, and layer controllability. Typically, single-crystal copper (Cu) foil is used to obtain single-crystal monolayer graphene. The graphene can be grown onto Cu foil under 1000° C. by CVD. Methane and hydrogen are used as precursor gases and argon as a carrier gas. Nickel can also be used as a substrate for CVD graphene growth. However, since the carbon atoms solubility of Ni is higher than Cu, the graphene growth on Ni is multilayer. FIG. 6C shows epitaxial growth graphene on SiC.

After CVD graphene growth, it can be transferred to an arbitrary substrate by various transfer techniques (e.g., wet transfer). Laser-induced-graphene (LIG), as shown in FIG. 61), is a novel method which does not require harsh conditions such as high ambient temperature, methane, and a hydrogen environment. Lin et al. reported LIG on a polyimide (PI) substrate, which is a cost-effective, one-step, and scalable approach for porous graphene film fabrication. They utilized a CO₂ laser(wavelength ˜10.6 μm) directly writing on the PI substrate, the energy of the laser irradiation can lead to an extremely high, but very localized, temperature, which can be up to 2,500° C. At this temperature, the C—O, C═O, and C—N bonds can be broken easily. Then, the oxygen and nitrogen will be recombined and released as gases, and the rest of the carbon atoms form aromatic compounds which rearrange to form porous graphene.

### Example 2—Methods for Graphene Oxide (GO) Synthesis

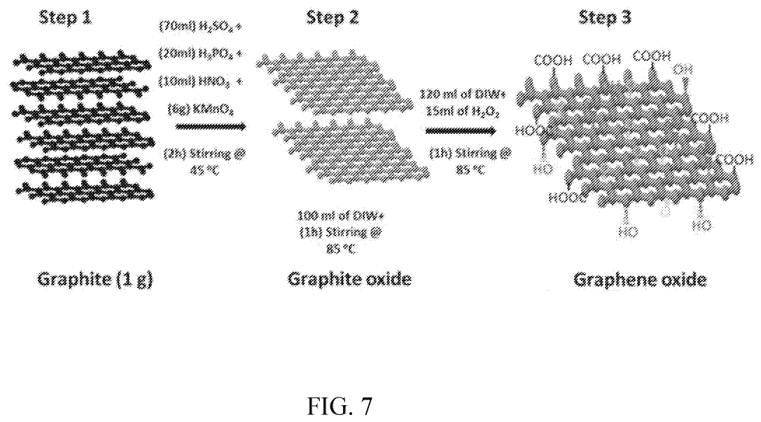

GO can be obtained by several different methods. The most commonly used is the Hummer's method, which was discovered in 1958 by Hummers and Offeman. In their work, they used graphite flake powder, potassium permanganate (KMnO₄), and sodium nitrate (NaNO₃) stirred into sulfuric acid to obtain GO. As applicable herein, this method has been slightly changed, called the modified Hummers method. The NaNO₃ is replaced with phosphoric acid (H₃PO₄) and nitric acid (HNO₃), incorporating H₂SO₄ as an intercalating agent. Then, hydrogen peroxide (H₂O₂) was added and with KMnO₄ as the oxidizing agents. FIG. 7 shows the synthesis process of GO: graphite flake powder was added into H₂SO₄, HNO₃, and H₃P₃O₄ mixture, then a 0° C. ice bath will be utilized to carefully control the temperature. KMnO₄ was added slowly during this time, followed by a 2 h oil bath at 45° C. Finally, deionized (DI) water with H₂O₂ was added, and the solution was centrifuged and washed with DIW until the solution reached a neutral pH.

### Example 3—Methods for Synthesis of Pyrolyzed Photoresist Films (PPF)

Advantageously, unlike certain other carbon-based nanomaterials, PPF does not require harsh or complicated fabrication steps. Instead, PPF can be obtained by annealing photoresist under high temperatures. Spin-coating a substrate with photoresist and patterning it to the desired shape allows one to achieve a film with a thickness related to the photoresist thickness and associated shrinkage during annealing. Film annealing is normally performed at between 500° C. and 1000° C. in a tube furnace with an inert environment, such as Ar, for one hour. As the photoresist starts heating, the color changes from transparent to black. The solvent and free phenols in the photoresist are evaporated out of the resist by the time the temperature reaches 300° C. A cross-linking reaction starts in the photoresist film when the temperature exceeds 300° C., whereby the C═O—H bonds will break and form C—O—C bonds. The bond reforming results in a significant thickness reduction, producing nanometer-scale films. Furthermore, PPF fabrication using a rapid thermal process (RTP) is possible, which indicates that the annealing temperature and the annealing time are more critical to fabricate PPF than the actual heating rate.

The following example describes a non-limiting method of fabricating an exemplary electrode device 110 of the present disclosure.

### Example 4—PPF Electrode Fabrication on α-SiC

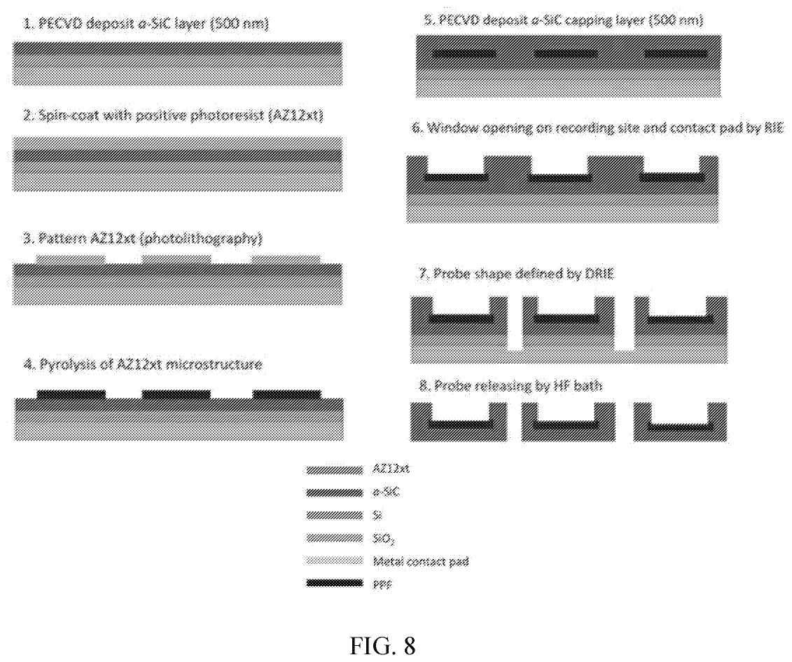

An α-SiC supported PPF electrode can be fabricated using standard semiconductor micromachining processes. First, a 500 nm thick α-SiC layer was deposited by plasma-enhanced chemical vapor deposition (PECVD) on a SiO₂/Si wafer. The deposition conditions are a platen temperature of 300° C., with a process pressure of 1100 mTorr, and 200 W of RF power. The precursors are CH₄ (200 sccm), %5 SiH₄ in H₂ (300 sccm), all diluted in 700 sccm He. FIG. 8 shows the fabrication process of single-ended electrodes with different recording site areas.

Positive photoresist (AZ12xt) was used to fabricate the devices. AZ12xt is spin-coated on the α-SiC wafer with 500 rpm for 20 s, then 3000 rpm for 50 s, and finally 11000 rpm for 2 s. The first spinning stage is to distribute the photoresist on the wafer uniformly, the second stage determines the thickness of the photoresist (in this case, 7.4 μm as measured with an optical profilometer), and the last stage is to eliminate any die edge effects.

The coated substrate was then soft-baked at 120° C. for 3 min, followed by UV exposure at an intensity of ˜120 mJ/cm² and a one-minute post-exposure bake at 95° C. Once the pattern has been transferred to the resist, the α-SiC wafer was then annealed in a tube furnace at different temperatures from 600° C. to 900° C. under an Ar and H₂ environment of 100 sccm and 5 sccm, respectively. The ramp-up rate was 10° C./min, followed by a long thermal soak at the desired temperature for an hour. Afterward, the wafer was cooled down to room temperature under an Ar and H₂ ambient.

To investigate the influence on recording area size, we fabricated four different recording area sized single-end electrodes with an area of 125 kμm², 7.8 kμm², 1.9 kμm², and 314 μm², respectively. This was followed by capping the electrodes with another layer of α-SiC (500 nm thickness) deposited by PECVD. The contact and electrode windows were then opened by RIE etching with 37 sccm CF₄ and 13 sccm O₂ for 20 min (both electrode tip and bonding pad areas were etched at the same time). To facilitate packaging 20 nm of Ti (adhesion layer) was e-beam evaporated followed by 150 nm of Au (without breaking vacuum) on the electrode site for packaging. To obtain Ohmic contact, the whole device is annealed at 500° C. in a rapid temperature process (RTP) furnace with a ramping rate of 10° C./s. Another Ti/Au metal layer was deposited on the annealed contacts to facilitate wire bonding, thus completing the electrode fabrication. The single-end electrode fabrication ends here, and the α-SiC/PPF neural probe will require another step to release the probe from the SiO₂ substrate by HF bath. This enables us to obtain free-standing α-SiC supported PPF-based neural probes.

### Example 5—PPF Electrode Fabrication on α-SiC

FIG. 8 shows a non-limiting technique for fabricating a pyrolyzed-photoresist-film (PPF) electrode (as shown in plan view labeled 0) on amorphous SiC. First, an α-SiC layer can be deposited (as shown in stage 1) on an electronic wafer (e.g., using plasma-enhanced chemical vapor deposition (PECVD) to deposit an α-SiC layer on a silicon wafer). This can be followed by patterning and pyrolyzing a photoresist film, as shown in stage 2. An α-SiC capping layer can then be deposited (e.g., via PECVD) as shown in stage 3, followed by opening of windows in the α-SiC film (e.g., via reactive-ion etching (RIE) to expose the recording and wire-bonding sites) as shown in stage 4 (e.g., openings in cap layer on left/right sides of depiction). The metal contact can then be deposited (e.g., by e-beam evaporation/lift off) as shown in stage 5, followed by defining the probe shape (e.g., using deep-reactive ion etching (DRIE) processes) as shown in stage 6. As described above, the probe shape may be adapted to suit a given application such as the uses described above. The sample can then be flipped and bonded with the handle wafer by crystal bond, as shown in stage 7. The backside of the silicon wafer can then be etched (e.g., by DRIE) as shown in stage 8. This can be followed by releasing the probe/finished device from the handle wafer by dissolving the crystal bond adhesion layer in an acid or solvent bath (e.g., an acetone bath or an HF bath), as shown in stage 9. A legend is also provided in FIG. 8 showing the compositions of the various layers and components shown in stages 0-9.

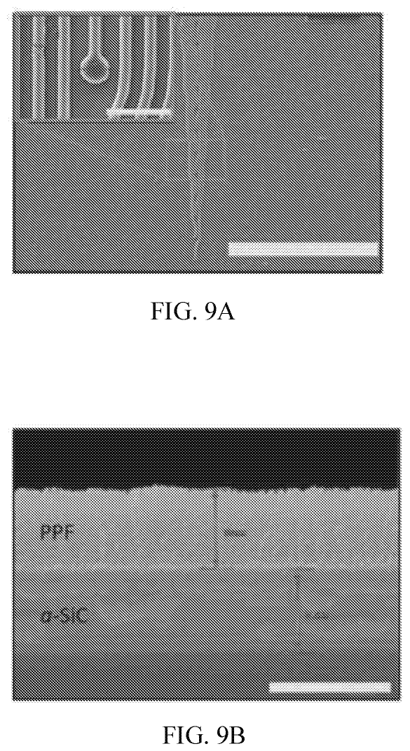

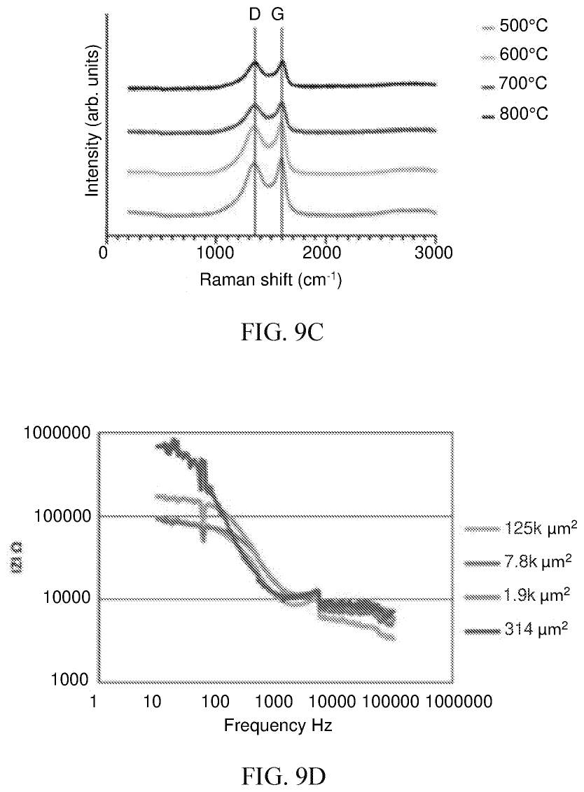

FIGS. 9A-D show preliminary data for the α-SiC supported PPF conductor neural probe. FIG. 9A shows a planar SEM image of 16 PPF conductor traces on one probe. The width of each trace and the gap between them is 10 μm with a 20 μm recording site as shown in the inset.

FIG. 9B is a cross-section SEM image of the PPF sample before annealing. The thickness of the top layer AZ12xt was 7.4 μm before annealing, and the inset image is after annealing under 700° C. for 1 h. The photoresist shrank from 7.4 μm to 332 nm or approximately 95% shrinkage after annealing.

Raman spectroscopy was used to investigate the crystalline and carbonization degree of the synthesized PPF versus annealing temperature and is shown in FIG. 9C. All of the PPF traces exhibited peaks at 1360 and 1580 cm⁻¹, which are called the D band (for disorder) and G band (for graphite), respectively. With increasing annealing temperature, the D band and G bands become slightly more distinguishable, which indicates that the PPF structure became more crystalline. Furthermore, the I_{D}/I_{G} ratio of annealed at 800° C. was ˜1, while at 500° C. ratio was ˜0.85. The observed ratio increase with temperature illustrates an increase in the material's structural order.

FIG. 9D shows preliminary electrochemical impedance spectroscopy (EIS) data from the PPF single-ended electrodes with different recording areas, ranging from 314 to 125 kμm². The impedance at the frequency of 1 kHz is critical since it is associated with the electrode recording efficiency due to 1 kHz being the neurons' spiking frequency. For all of our PPF electrodes, the impedance at 1 kHz is about 10 kΩ, which is an order of magnitude lower than for Pt electrode with the same recording area. This result indicates the PPF has an excellent impedance range for neural recording applications.

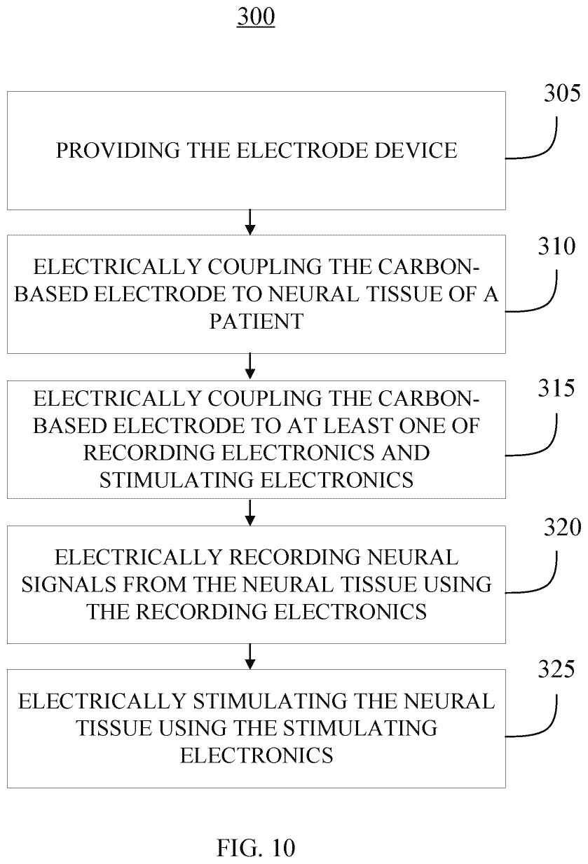

As shown in FIG. 10, the present disclosure also provides a method 300 of using an implantable neural interface/neural probe. The method 300 includes the steps of providing the electrode device 100 (step 305); electrically coupling the carbon-based electrode 125 to neural tissue of a patient (step 310); and electrically coupling the carbon-based electrode 125 to at least one of recording electronics and stimulating electronics (step 315). The recording electronics are configured to electrically record neural signals from the neural tissue of the patient, whereas the stimulating electronics are configured to electrically stimulate the neural tissue of the patient.

The metal portion of the device (i.e., the metal contact pad) is only used on the bond pad for packaging the neural probe. Thus, when the probe of the present disclosure is used, the material that contacts the brain is only SiC and carbon (e.g., PPF). Advantageously, no metals or polymers contact the brain, which could stimulate an immune response and degrade in the brain's harsh environment over time.

In some embodiments, the carbon-based electrode 125, when implanted into a patient, penetrates into neural tissue of the patient. One embodiment favors the thin film, planar penetrating probe which was first developed at the University of Michigan in the 1970's. This style of penetrating probe is delivered directly into brain tissue or nerve bundles using needles containing the electrodes fashioned on their surfaces. Stereotaxic, three dimensional frames with micromanipulators are used for precise implantation into the tissue. The thin-film electrode enables customization of the probe shape, number of needles, and number of electrodes so the device can precisely target specific regions of interest for recording or stimulation.

In some embodiments, method 300 includes the step 320 of electrically recording neural signals from the neural tissue using the recording electronics. This allows the carbon-based electrode 125 to measure electrical signals from the brain (e.g., action potentials). Recording electronics that can be used with the electrode device of the present disclosure include any type of low voltage signal acquisition electronics. Intan Technologies has a widely used chipset for many manufacturers, like NeuroNexus and Plexon, while World Precision Instruments provides an analog approach.

In some cases, method 300 includes electrically stimulating the neural tissue of the patient using the stimulating electronics (step 325). Applying a signal to the electrodes can be achieved using either potentiostatic or galvanostatic electronics. Potentiostatic electronics involve applying a constant voltage, while galvanostatic electronics involve applying a constant current. Constant current is typically preferred as current is believed to be the quantity that activates stimulation, and the feedback system adjusts the voltage to compensate for changes in the resistance to maintain a constant current flow.

In some cases, both recording 320 and stimulating 325 steps are performed. In other cases, the recording step 320 is performed, but the stimulating step 325 is not. In other cases, the stimulating step 325 can be performed, but the recording step 320 is not.

In certain embodiments, the neural interface of the present disclosure is electrically coupled to an electrode amplifier. As is well-known in the art, an electrode amplifier is a circuit that allows the output of the electrode to be monitored by an interface. In more detail, the electrode amplifier amplifies the voltage produced by an electrode into a range where it can be monitored by the lab interface. For the carbon-based electrode, the desired amplified voltage ranges from microvolt-level signals up to about 2 volts.

In certain embodiments, the neural interface is electrically coupled to a multiplexer. A multiplexer is a combinational logic circuit that acts as a switcher for multiple inputs to a single common output line. Thus, a multiplexer allows one signal selected from several inputs to be connected to a single output.

A system can be provided for stimulating and recording inputs/outputs from electrodes as described above. For example, and as described in more detail below, such a system may comprise the stimulating electronics and recording electronics, a processor for controlling the same, as well as memory for storing readings from, and instructions for stimulating, such electrodes.

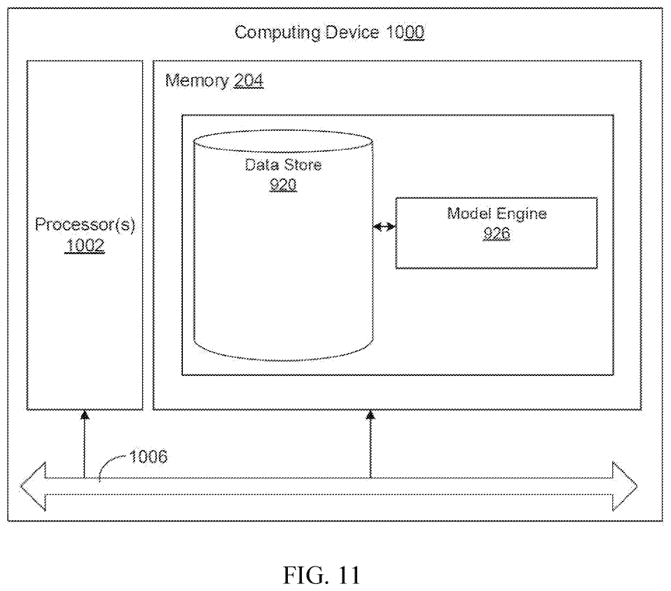

FIG. 11 illustrates an example schematic block diagram of a computing device 1000 for a computing environment according to various embodiments described herein. The computing device 1000 includes at least one processing system, for example, having a processor 1002 and a memory 204, both of which are electrically and communicatively coupled to a local interface 1006. The local interface 1006 can be embodied as a data bus with an accompanying address/control bus or other addressing, control, and/or command lines.

In various embodiments, the memory 204 stores data and software or executable-code components executable by the processor 1002. For example, the memory 204 can store executable-code components associated with software instructions 926 for execution by the processor 1002. The memory 204 can also store data stored in a data store 920, among other data.

Data store 920 may be a large database comprising data utilized for a variety of purposes, implemented via local network storage (e.g., on-site at a facility, research institution, or hospital) or cloud storage. Data store 920 may be in communication with one or more remote servers, in which case the computing environment simply retrieves data on an as-needed basis from the remote data services. Data store 920 may comprise data such as medical records, as well as previous electrode recordings and results from stimulating the electrodes. In some embodiments, the data store 920 may be an aggregation of data obtained from research institutions or hospitals that collect data from patients.

It is noted that the memory 204 can store other executable-code components for execution by the processor 1002. For example, an operating system can be stored in the memory 204 for execution by the processor 1002. Where any component discussed herein is implemented in the form of software, any one of a number of programming languages can be employed such as, for example, C, C++, C #, Objective C, JAVA®, JAVASCRIPT®, Perl, PHP, VISUAL BASIC®, PYTHON®, RUBY, FLASH®, or other programming languages.

As discussed above, in various embodiments, the memory 204 stores software for execution by the processor 1002. In this respect, the terms “executable” or “for execution” refer to software forms that can ultimately be run or executed by the processor 1002, whether in source, object, machine, or other form. Examples of executable programs include, for example, a compiled program that can be translated into a machine code format and loaded into a random access portion of the memory 204 and executed by the processor 1002, source code that can be expressed in an object code format and loaded into a random access portion of the memory 204 and executed by the processor 1002, or source code that can be interpreted by another executable program to generate instructions in a random access portion of the memory 204 and executed by the processor 1002, etc.

An executable program can be stored in any portion or component of the memory 204 including, for example, a random access memory (RAM), read-only memory (ROM), magnetic or other hard disk drive, solid-state, semiconductor, universal serial bus (USB) flash drive, memory card, optical disc (e.g., compact disc (CD) or digital versatile disc (DVD)), floppy disk, magnetic tape, or other types of memory devices.

In various embodiments, the memory 204 can include both volatile and nonvolatile memory and data storage components. Volatile components are those that do not retain data values upon loss of power. Nonvolatile components are those that retain data upon a loss of power. Thus, the memory 204 can include, for example, a RAM, ROM, magnetic or other hard disk drive, solid-state, semiconductor, or similar drive, USB flash drive, memory card accessed via a memory card reader, floppy disk accessed via an associated floppy disk drive, optical disc accessed via an optical disc drive, magnetic tape accessed via an appropriate tape drive, and/or other memory component, or any combination thereof. In addition, the RAM can include, for example, a static random access memory (SRAM), dynamic random access memory (DRAM), or magnetic random access memory (MRAM), and/or other similar memory device. The ROM can include, for example, a programmable read-only memory (PROM), erasable programmable read-only memory (EPROM), electrically erasable programmable read-only memory (EEPROM), or other similar memory device.

The processor 1002 can be embodied as one or more processors 1002 and the memory 204 can be embodied as one or more memories 204 that operate in parallel, respectively, or in combination. Thus, the local interface 1006 facilitates communication between any two of the multiple processors 1002, between any processor 1002 and any of the memories 204, or between any two of the memories 204, etc. The local interface 1006 can include additional systems designed to coordinate this communication, including, for example, a load balancer that performs load balancing.

The software instructions 926 may comprise instructions for controlling the stimulating and recording electronics discussed above. For example, the software instructions may contain parameters for adjusting the stimulating signal attributes to be applied through electrodes, or may contain instructions for receiving, digitizing, and recording signals received through electrodes. The software instructions 926 can be embodied, at least in part, by software or executable-code components for execution by general purpose hardware. Alternatively, the same can be embodied in dedicated hardware or a combination of software, general, specific, and/or dedicated purpose hardware. If embodied in such hardware, each can be implemented as a circuit or state machine, for example, that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

Also, any logic or application described herein (including the software instructions 926) that are embodied, at least in part, by software or executable-code components, can be embodied or stored in any tangible or non-transitory computer-readable medium or device for execution by an instruction execution system such as a general purpose processor. In this sense, the logic can be embodied as, for example, software or executable-code components that can be fetched from the computer-readable medium and executed by the instruction execution system.

The computer-readable medium can include any physical media such as, for example, magnetic, optical, or semiconductor media. More specific examples of suitable computer-readable media include, but are not limited to, magnetic tapes, magnetic hard drives, memory cards, solid-state drives, USB flash drives, or optical discs. Also, the computer-readable medium can include a RAM including, for example, an SRAM, DRAM, or MRAM. In addition, the computer-readable medium can include a ROM, a PROM, an EPROM, an EEPROM, or other similar memory device.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The computing environment can be implemented so as to perform one or more of a variety of functions for different types of users, through implementation of the models and algorithms described above. For example, the system can provide services to a facility, clinic, or hospital. The facility, hospital, or other user may upload software for stimulating electrodes, as well as for recording signals from electrodes. Then the system may process the data using the software instructions 926. Further, the software instructions 926 may make recommendations for the patient regarding treatment plans (e.g., for treating or diagnosing a nervous system disease).

It should be noted that, as used herein, the term mechanism can encompass hardware, software, firmware, or any suitable combination thereof.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. An electrode device comprising:
a base layer comprising silicon carbide;
an intermediate layer located over the base layer, the intermediate layer comprising a carbon-based electrode; and
a capping layer located over the base layer and partially surrounding the carbon-based electrode, the capping layer comprising silicon carbide.

2. The electrode device of claim 1, wherein the carbon-based electrode comprises graphene, graphene oxide, reduced graphene oxide, carbon nanotubes, or a pyrolyzed-photoresist-film.

3. The electrode device of claim 1, wherein the carbon-based electrode comprises a pyrolyzed-photoresist-film.

4. The electrode device of claim 1, wherein the silicon carbide of both the base layer and the capping layer comprises amorphous silicon carbide.

5.-7. (canceled)

8. The electrode device of claim 1, wherein the base layer has a thickness in a range of from 0.5 μm to 5 μm, the capping layer has a thickness in a range of from 0.5 μm to 5 μm, or and the intermediate layer has a thickness in a range of from 0.1 μm to 1 μm.

9. The electrode device of claim 1, further comprising a metal contact pad electrically coupled to the carbon-based electrode, the metal contact pad being exposed through an opening in the capping layer.

10. A method of making an electrode device comprising:
depositing a base layer onto an electronic wafer, the base layer comprising silicon carbide;
forming an intermediate layer on the base layer, the intermediate layer comprising a carbon-based electrode;
depositing a metal layer onto carbon traces, the metal layer comprising a metal contact pad, the carbon traces being configured to electrically couple the metal contact pad to the carbon-based electrode;
depositing a capping layer onto the intermediate layer, the capping layer comprising silicon carbide; and
forming one or more openings in the capping layer to expose at least a portion of the carbon-based electrode and at least a portion of the metal contact pad.

11. The method of claim 10, wherein the step of forming an intermediate layer on the base layer comprises:
depositing a photoresist onto the base layer and patterning the photoresist to a desired shape; and
pyrolyzing the photoresist so as to convert the photoresist to a pyrolyzed photoresist film.

12. The method of claim 10, further comprising a step of releasing the electronic wafer from the base layer by subjecting the electronic wafer to an acid bath or a solvent bath.

13. The method of claim 10, wherein the carbon-based electrode comprises graphene, graphene oxide, reduced graphene oxide, or carbon nanotubes.

14.-16. (canceled)

17. The method of claim 10, wherein the base layer has a thickness in a range of from 0.5 μm to 5 μm, the capping layer has a thickness in a range of from 0.5 μm to 5 μm, or and the intermediate layer has a thickness in a range of from 0.1 μm to 1 μm.

18. The method of claim 10, wherein the silicon carbide of both the base layer and the capping layer comprises amorphous silicon carbide.

19. A method of using an implantable neural interface, the method comprising:
providing an electrode device comprising:
a base layer comprising silicon carbide;
an intermediate layer located over the base layer, the intermediate layer comprising a carbon-based electrode; and
a capping layer located over the base layer and partially surrounding the carbon-based electrode, the capping layer comprising silicon carbide;
electrically coupling the carbon-based electrode to neural tissue of a patient;
electrically coupling the carbon-based electrode to at least one of recording electronics and stimulating electronics, wherein the recording electronics are configured to electrically record neural signals from the neural tissue and the stimulating electronics are configured to electrically stimulate the neural tissue.

20. The method of claim 19, further comprising electrically recording neural signals from the neural tissue using the recording electronics.

21. The method of claim 19, further comprising electrically stimulating the neural tissue using the stimulating electronics.

22. The method of claim 19, wherein the carbon-based electrode comprises graphene, graphene oxide, reduced graphene oxide, carbon nanotubes, or a pyrolyzed-photoresist-film.

23. The method of claim 19, wherein the carbon-based electrode comprises a pyrolyzed-photoresist-film.

24. The method of claim 19, wherein the silicon carbide of both the base layer and the capping layer comprises amorphous silicon carbide.

25.-27. (canceled)

28. The method of claim 19, wherein the base layer has a thickness in a range of from 0.5 μm to 5 μm, the capping layer has a thickness in a range of from 0.5 μm to 5 μm, or and the intermediate layer has a thickness in a range of from 0.1 μm to 1 μm.

29. The method of claim 19, wherein the silicon carbide of both the base layer and the capping layer comprises amorphous silicon carbide.

30. The electrode device of claim 1, further comprising a metal contact pad electrically coupled to the carbon-based electrode, the metal contact pad being exposed through an opening in the capping layer,
wherein the carbon-based electrode comprises a pyrolyzed-photoresist-film;
wherein the silicon carbide of both the base layer and the capping layer comprises amorphous silicon carbide; and
wherein the base layer has a thickness in a range of from 0.5 μm to 5 μm, the capping layer has a thickness in a range of from 0.5 μm to 5 μm, and the intermediate layer has a thickness in a range of from 0.1 μm to 1 μm.
